(19) United States
(12) Patent Application Publication
(10) Pub. No.: US 2023/0338309 A1
(43) Pub. Date: Oct. 26, 2023

(54) COMPOSITIONS AND METHODS FOR TOPICAL TREATMENT OF VASCULAR CONDITIONS

(71) Applicant: JSS RESEARCH, LLC, Pinecrest, FL (US)

(72) Inventor: Spencer Bouhadir, Lake Worth, FL (US)

(21) Appl. No.: 18/340,702

(22) Filed: Jun. 23, 2023

Related U.S. Application Data
(63) Continuation of application No. PCT/US2022/082648, filed on Dec. 30, 2022.
(60) Provisional application No. 63/394,995, filed on Aug. 4, 2022.
(60) Provisional application No. 63/295,579, filed on Dec. 31, 2021.

(51) Int. Cl. A61K 31/137; A61K 31/455; A61K 47/36; A61K 9/00; A61K 47/22; A61P 9/14; A61P 17/00; A61K 47/10; A61K 47/18; A61K 47/08

(52) U.S. Cl. CPC A61K 31/137; A61K 9/0014; A61K 31/455; A61K 47/08; A61K 47/10; A61K 47/183; A61K 47/22; A61K 47/36; A61P 9/14; A61P 17/00

(57) ABSTRACT

The present disclosure provides, inter alia, formulations for topical treatment of vascular conditions. In certain embodiments, the formulations comprise a delivery system enabling penetration of sympathomimetic agents to the dermis where vasculature including veins and arteries may be treated. The present disclosure also provides methods for treating vascular conditions for which effective topical treatment is currently inadequate or not available. Methods for making such formulations are also provided.

### CROSS REFERENCE TO RELATED APPLICATIONS

The present application claims benefit of U.S. Provisional Pat. Application Serial No. 63/295,579, filed on Dec. 31, 2021 and U.S. Provisional Pat. Application Serial No. 63/394,995, filed on Aug. 4, 2022, which applications are incorporated by reference herein in their entireties.

### BACKGROUND

### Field

This disclosure relates generally to therapeutic and cosmetic formulations and their methods of use. More specifically, this disclosure is related to formulations that may be topically applied for treatment of vascular conditions.

### Description of Related Art

Adrenaline is an essential hormone and neurotransmitter in human physiology. It is a member of the catecholamine family of compounds. Catecholamines are a group of amines with a 3,4-dihydroxy-substituted phenyl ring that are constructed on a phenethylamine skeleton containing at least one phenolic hydroxy- group, an alcohol hydroxy- group, and an N-methylated amino-group. Catecholamines are non-selective agonists of adrenergic receptors whose four subtypes are ubiquitous throughout vertebrate tissue. Structurally related molecules such as phenylephrine and ephedrine can also activate adrenergic receptors. A predominant physiological effect of stimulating adrenergic receptors is vasoconstriction, which concentrates blood in major muscles in response to acute stress (i.e., fight-or-flight response).

As pharmaceuticals, catecholamines and some of their structural relatives are known as sympathomimetic drugs. Their mechanism primarily involves stimulating sympathetic nerves to induce vasoconstriction and increase blood pressure. Sympathomimetic drugs, primarily epinephrine, are used in decongestants as a nasal spray and an oral tablet. Intramuscular injection of epinephrine is a standard treatment for allergic emergencies. Phenylephrine is the active ingredient in topically applied hemorrhoid treatments. Sympathomimetics may also be given as an ophthamalic drop to dilate the pupil for diagnostic procedures. In acute contexts, sympathomimetic drugs are used intravenously to treat cardiac arrest, low blood pressure, and to delay childbirth. They are also applied topically during some surgeries to control local bleeding.

In topical use, sympathomimetic drugs are most effective on mucous membranes. Mucous membranes, include the eyes, ears, inside the nose, inside the mouth, lip, vagina, the urethral opening and the anus, and are locations in skin where the overlying epidermis is thin and the underlying dermis is at or near the surface. Sympathomimetic drugs are effective at mucous membranes because they readily come into direct contact with vasculature where they have vasoconstrictive effect.

Everywhere else, however, topical application of sympathomimetics to skin is less effective because most vasculature is located at least 2 mm below the skin surface and because skin is nearly waterproof. Because catecholamines and related compounds are electronegative, water-soluble substances, they are excluded from absorption by skin with high efficiency.

Nevertheless, topically applied sympathomimetic drugs could be a preferable treatment modality for some conditions where topical treatment is not currently an option or not sufficiently effective. Some skin and/or vasculature conditions that have been considered for treatment by topical sympathomimetics include localized fat elimination, rosacea, varicose veins and alopecia. Topical sympathomimetics have also been used in attempts to localize or exclude other drugs from skin, such as with chemotherapeutics to prevent hair loss. Some of these approaches have also sought to enhance topical sympathomimetic effectiveness by increasing skin penetration.

Several transdermal carriers and surfactants have been proposed for use with sympathomimetics including polyethylene glycol, propylene glycol, diethylene glycol monoethyl etheralcohol, caprylic acid, caproic acid, capric glycerides, oleic acid, lauric acid, isopropyl myristate, triethanolamine, lecithin, dimethylsulfoxide. diisopropyl adipate, polymethacrylic acid, carbopol, acetone and tween 80. Although effective to varying degrees in increasing penetration depth, many of these carriers and enhancers have undesirable side effects including skin irritation, and/or toxicity resulting from excessive penetration and uptake into the blood. Furthermore, many of these carriers are incompatible with the solubility of active ingredients in the transdermal carrier formulation. Even with a highly effective penetration agent, poor solubility of the active ingredient results in agglomeration or undesirably large particle size and reduced penetration efficiency.

As a result of these obstacles and limitations, there are no economical, convenient, and effective formulations enabling topical treatment of vascular conditions with sympathomimetics outside of the mucous membranes. Thus, there exists a need for enhanced formulations for topical application of sympathomimetics to skin as a strategic treatment modality that provides a preferable alternative to currently available modalities such as, e.g., surgical intervention, high-energy intervention, and systemic pharmaceutical and/or cosmetic treatments.

### SUMMARY OF THE DISCLOSURE

For purposes of summarizing the disclosure and the advantages achieved over the prior art, certain objects and advantages of the disclosure are described herein. Not all such objects or advantages may be achieved in any particular embodiment. Thus for example, those skilled in the art will recognize that the disclosure may be embodied or carried out in a manner that achieves or optimizes one advantage or group of advantages as taught herein without necessarily achieving the other objects or advantages has may be taught or suggested herein.

According to some aspects, the present disclosure provides a formulation for topical treatment of a vascular condition in a subject, the formulation comprising: a sympathomimetic agent capable of effecting vasoconstriction in the skin of the subject; a delivery system that is effective to transport an amount of the sympathomimetic agent through the epidermis that is effective to produce vasoconstriction in the skin of the subject; and an aqueous component.

In certain embodiments, the sympathomimetic agent capable of effecting vasoconstriction is selected from the group consisting of epinephrine, norepinephrine, phenylephrine, synephrine, ephedrine, or combinations thereof. In certain embodiments, the sympathomimetic agent for vasoconstriction is present in a concentration of about 0.1 wt% to 20 wt%.

In certain embodiments, the delivery system as disclosed herein comprises a composition selected from the group consisting of polyethylene glycol (PEG), ethoxydiglycol (EDG), diethylene glycol monoethyl ether (“Transcutol”), dimethyl isosorbide (DMI), dimethyl sulfoxide (DMSO), sodium lauryl sulfate (SDS), Polyoxyethylene (20) sorbitan monooleate (Tween 80) or combinations thereof. In certain embodiments, the delivery system further comprises a composition selected from the group consisting of propanediol, hyaluronic acid, sodium hyaluronate, glycerin, propylene glycol, caprylyl glycol, butylene glycol, pentylene glycol, sodium carbomer, horse chestnut extract, aescin, vanillin, xanthan gum, hydroxypropyl methylcellulose, Disodium EDTA, Tetrasodium EDTA, phenoxyethanol, ethylhexylglycerin, benzyl alcohol, diazolidinyl urea, iodopropynyl butylcarbamate DMDM hydantoin, paraben, propyl paraben, methylparaben, and polyacrylate crosspolymer or combinations thereof. In certain embodiments, the delivery system is present at a concentration of about 0.1 wt% to 20 wt% of the formulation. In certain embodiments, the delivery system is present at a concentration of at about 1 wt% to 60 wt% of the formulation.

According to certain embodiments, the formulations disclosed herein comprise an aqueous component present at a concentration of about 1 wt% to 60 wt%.

According to certain embodiments, the formulations disclosed herein are effective to topically treat or ameliorate a condition that is responsive to vasoconstriction. In certain embodiments, the formulation is effective to topically treat one or more of hemorrhoids, varicose veins, spider veins, rosacea and periorbital edema.

According to some aspects, the present disclosure provides a method of topically treating a vascular condition comprising the step of contacting skin with a formulation as disclosed herein. According to certain embodiments of the treatment methods disclosed herein, the epidermis is penetrated by the sympathomimetic agent to a depth of between about 1-5 millimeters. In certain embodiments, the formulations disclosed herein are applied twice daily for about 20 days for treatment of hemorrhoids. According to certain embodiments, the formulations disclosed herein are applied once daily for about 30 days for treatment of spider veins. According to certain embodiments, the formulations disclosed herein are applied once daily for about 35 days for treatment of varicose veins. According to certain embodiments, the formulations disclosed herein are applied serially as needed for treatment of rosacea. According to certain embodiments, the formulations disclosed herein are applied serially as needed for treatment of periorbital edema.

According to some aspects, the present disclosure provides a method of preparing a formulation for topical treatment of a vascular condition comprising the steps of: (i) dissolving a sympathomimetic agent in a niacinamide serum; (ii) combining the product of step (i) with a penetration enhancing agent; (iii) combining the product of step (ii) with a humectant and emollient; (iv) combining the product of step (iii) with a preservative agent; and (v) combining the product of step (iv) with a hydrated hyaluronic acid. In certain embodiments, the methods disclosed herein further comprise the step of (vi) stirring the product of step (v) vigorously until a clear, slightly viscous solution is obtained. In certain embodiments, the hyaluronic acid is partially hydrated prior to addition to the formulation. In certain embodiments, the amount of sympathomimetic dissolved in the formulation for treatment of periorbital edema is at a concentration of least about 0.1 wt% and at most about 5 wt%.

In certain embodiments, the vascular condition topically treatable is selected from the group consisting of varicose veins, spider veins, hemorrhoids, rosacea, and periorbital edema.

In certain embodiments, topical sympathomimetics applied in combination with a delivery system provides more effective hemorrhoid treatment.

In certain embodiments, topical sympathomimetics applied in combination with a delivery system provides a previously unavailable treatment of varicose veins, spider veins, rosacea and periorbital edema.

In one aspect, the composition for topical treatment of certain vascular conditions is disclosed. The composition comprises components selected from the group consisting of an active ingredient, a delivery system, and a carrier system.

In certain embodiments, the active ingredient may be a sympathomimetic selected from the group consisting of epinephrine, norepinephrine, phenylephrine, synephrine, ephedrine, and combinations thereof.

In another aspect, a method of making a composition for topical treatment of certain vascular conditions is disclosed.

In another aspect, a method of using a topically applied composition for treatment and/or prevention of certain vascular conditions is disclosed.

In certain embodiments, the present disclosure provides a formulation for topical treatment of a vascular condition in a subject, the formulation comprising: 0.1 wt% Disodium EDTA, 8 wt% Niacinamide, 10 wt% p-Synephrine HCL, 3 wt% Glycerin, 10 wt% Butylene Glycol, 10 wt% Ethoxydiglycol, 0.5 wt% Sodium Carbomer, 0.8 wt% Benzyl Alcohol, and 0.1 wt% Ethylhexylglycerin.

In certain embodiments, the present disclosure provides a formulation for topical treatment of a vascular condition in a subject, the formulation comprising: 4 wt% Epinephrine, 8 wt% Dimethyl isosorbide, 10 wt% Niacinamide, 5 wt% Glycerin, 1 wt% Vanillin, 10 wt% Propylene Glycol, 0.1 wt% Tetrasodium EDTA, 0.2 wt% diazolidinyl urea, and 0.4 wt% Xanthan gum.

In certain embodiments, the present disclosure provides a formulation for topical treatment of a vascular condition in a subject, the formulation comprising: 12 wt% D-Synephrine HCL, 15 wt% Dimethyl isosorbide, 1 wt% Vanillin, 2 wt%Glycerin, 6 wt% Pentylene glycol, 0.7 wt% Benzyl alcohol, 0.1 wt% Disodium EDTA, and 0.6 wt% Sodium hyaluronate.

In certain embodiments, the present disclosure provides a formulation for topical treatment of a vascular condition in a subject, the formulation comprising: 6 wt% L-Synephrine, 6 wt% Dimethyl isosorbide, 8 wt% Niacinamide, 1 wt% Glycerin, 8 wt% Butylene glycol, 0.05 wt% Disodium EDTA, 0.7 wt% Hyaluronic acid, 0.3 wt% Propylparaben, 0.5 wt% Methylparaben.

In certain embodiments, the present disclosure provides a formulation for topical treatment of a vascular condition in a subject, the formulation comprising: 8 wt% Synephrine, 10 wt% Dimethyl isosorbide, 8 wt% Niacinamide, 2 wt% Glycerin, 10 wt% Propanediol, 0.6 wt% Phenoxyethanol, 0.4 wt% Caprylyl glycol, 0.05 wt% Disodium EDTA, and 0.5 wt% hydroxypropyl methylcellulose.

In certain embodiments, the present disclosure provides a formulation for topical treatment of a vascular condition in a subject, the formulation comprising: 5 wt% Synephrine, 8 wt% Ethoxydiglycol, 4 wt% Niacinamide, 4 wt% Glycerin, 0.3 wt% Caprylyl glycol, 0.9 wt% Phenoxyethanol, 10 wt% Propanediol, 0.1 wt% Disodium EDTA, and 0.6 wt% polyacrylate crosspolymer.

In certain embodiments, the present disclosure provides a formulation for topical treatment of a vascular condition in a subject, the formulation comprising: 0.1 wt% Disodium EDTA, 8 wt% Niacinamide, 10 wt% p-Synephrine HCL, 3 wt% Glycerin, 10 wt% Butylene Glycol, 10 wt% Ethoxydiglycol, 0.5 wt% Sodium Carbomer, 0.8 wt% Benzyl Alcohol, and 0.2 wt% DMDM Hydantoin.

In certain embodiments, the present disclosure provides a formulation for topical treatment of a vascular condition in a subject, the formulation comprising: 4 wt% Epinephrine, 8 wt% Dimethyl isosorbide, 10 wt% Niacinamide, 5 wt% Glycerin, 1 wt% Vanillin, 10 wt% Propylene Glycol, 0.1 wt% Tetrasodium EDTA, 0.2 wt% DMDM hydantoin, and 0.4 wt% Xanthan gum.

In certain embodiments, the present disclosure provides a formulation for topical treatment of a vascular condition in a subject, the formulation comprising: 0.05 wt% Disodium EDTA, 8 wt% Niacinamide, 5 wt% Synephrine HCL, 10 wt% Dimethyl isosorbide, 10 wt% Propanediol, 3 wt% Glycerin, 0.6 wt% Phenoxyethanol, 0.1 wt% Caprylyl glycol, and 0.8 wt% Hyaluronic acid.

In certain embodiments, the present disclosure provides a formulation for topical treatment of a vascular condition in a subject, the formulation comprising: 15 wt% P-Synephrine HCL, 15 wt% Dimethyl Isosorbide, 10 wt% Propanediol, 8 wt% Niacinamide, 0.3 wt% Caprylyl Glycol, 0.7 wt% Phenoxyethanol, 0.05 wt% Disodium EDTA, and 0.5 wt% Hyaluronic acid.

### BRIEF DESCRIPTION OF THE DRAWINGS

FIG. 1 discloses the chemical structure of representative active ingredients suitable for use in certain embodiments of the topically applied sympathomimetic compositions disclosed herein.

FIG. 2 discloses a graphic depiction of a representative process for preparing a topically applied sympathomimetic composition according to certain embodiments disclosed herein.

FIG. 3 discloses the results of treatment efficacy experiments on hemorrhoids using a topically applied sympathomimetic composition according to certain embodiments disclosed herein.

FIG. 4 discloses the results of treatment efficacy experiments on spider veins using a topically applied sympathomimetic composition according to certain embodiments disclosed herein.

FIG. 5 discloses the results of treatment efficacy experiments on varicose veins using a topically applied sympathomimetic composition according to certain embodiments disclosed herein.

FIG. 6 depicts the results of treatment efficacy experiments on hemorrhoids using a topically applied sympathomimetic composition according to certain embodiments disclosed herein in comparison to treatment with PREPARATION-H™ and placebo.

FIG. 7 depicts the results of treatment efficacy experiments on varicose veins or spider veins using a topically applied sympathomimetic composition according to certain embodiments disclosed herein in comparison to treatment with PREPARATION-H™ and placebo.

FIG. 8 depicts the results of treatment efficacy experiments on varicose veins or spider veins using a topically applied sympathomimetic composition according to certain embodiments disclosed herein in comparison to treatment with PHARMAPULSE™ and placebo.

### DETAILED DESCRIPTION

According to some aspects, the present disclosure provides compositions and methods for topical treatment of vascular conditions by increasing the epidermal penetration depth of sympathomimetic drugs. In certain embodiments, a composition comprising skin penetration agents and/or surfactants enables penetration of sympathomimetic drugs through layers of skin down to the dermis and underlying vasculature without absorption into the blood stream. In certain embodiments, the compositions, methods, and kits disclosed herein enable penetration of sympathomimetics to a depth of up to 5 millimeters (mm) in human skin, where the amount of sympathomimetic penetrated can effect vasoconstriction.

### Formulations

According to some aspects, the present disclosure provides a formulation for topical treatment of a vascular condition in a subject, the formulation comprising: a sympathomimetic agent capable of effecting vasoconstriction in the skin of the subject; a delivery system that is effective to transport an amount of the sympathomimetic agent through the epidermis that is effective to produce vasoconstriction in the skin of the subject; and an aqueous component.

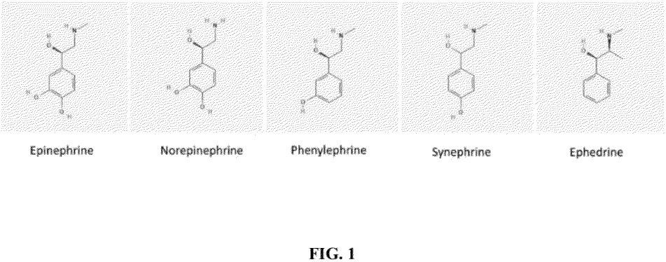

As disclosed herein a “sympathomimetic agent” means any agent that acts as a stimulant compound mimicking the effects of endogenous agonists of the sympathetic nervous system. The sympathomimetic agent as disclosed herein can be direct acting (i.e., direct interaction between agent and cell receptor), such as such as α-adrenergic agonists, β-adrenergic agonists, and dopaminergic agonists, or can be indirect-acting (i.e., no interaction between drug and cell receptor), such as monoamine oxidase inhibitors (MAOIs), catechol-o-methyltransferase (COMT) inhibitors, release stimulants, and reuptake inhibitors that increase the levels of endogenous catecholamines. Sympathomimetic agents according to certain embodiments disclosed herein include, but are not limited to, epinephrine (4-[(1R)-1-hydroxy-2-(methylamino)ethyl]benzene-1,2-diol), norepinephrine (4-[(1R)-2-amino-1-hydroxyethyl]benzene-1,2-diol), phenylephrine (3-[(1R)-1-hydroxy-2-(methylamino)ethyl]phenol), synephrine (4-[1-hydroxy-2-(methylamino)ethyl]phenol), ephedrine ((1R,2S)-2-(methylamino)-1-phenylpropan-1-ol), all enantiomers and structural isomers thereof or combinations thereof. See FIG. 1.

In certain embodiments, the composition may comprise a sympathomimetic active ingredient or combinations thereof. In certain embodiments, the active ingredient component of the formulation comprises, comprises about, comprises at most, or comprises at most about 20 wt%, 15 wt%, 10 wt% or 5 wt%, relative to the rest of the components of the formulation, or any range of values therebetween and comprises, comprises about, comprises at least, or comprises at least about 1 wt%, 0.5 wt%, 0.1 wt%, or any range of values therebetween.

In certain embodiments, the composition may comprise a delivery system. In certain embodiments, the delivery system may comprise a penetration-enhancing agent and/or surfactant ingredient or combinations thereof. In certain embodiments, the delivery system is effective to transport the sympathomimetic agent through the skin to contact blood vessels. In certain embodiments, the delivery system is effective to transport an amount of sympathomimetic agent into contact with blood vessels such that the sympathomimetic agent causes the smooth muscle in the blood vessels to constrict, i.e., result in vasoconstriction. As used herein, “vasoconstriction” means a contraction of blood vessels making the lumen of the blood vessel narrower.

In certain embodiments, the formulations disclosed herein are effective to improve a vascular condition as measured by the Clinical, Etiological, Anatomical, and Pathophysiological (CEAP) classification system, which grades a vascular condition from a clinical category of C0 (no visible or palpable signs of disease) to a clinical category of C6 (active ulcer). In certain embodiments, the formulations disclosed herein are effective to improve the clinical category of a vascular condition by at least one category (e.g., C1 to C0), at least two clinical categories (e.g., C2 to C0), at least three clinical categories (e.g., C3 to C0), at least four clinical categories (e.g., C4 to C0), at least five clinical categories (e.g., C5 to C0), or at least 6 clinical categories (e.g., C6 to C0).

In certain embodiments, the formulations disclosed herein are effective to improve rosacea as measured by the standard grading system for rosacea, which grades the condition from absent, mild, moderate, or severe (grades 0-3, respectively). In some embodiments, the formulations disclosed herein are effective to improve the clinical grade of rosacea by at least one grade (e.g., grade 1 to grade 0), at least two grades (e.g., grade 2 to grade 0), or at least three grades (e.g., grade 3 to grade 0).

In certain embodiments, the formulations disclosed herein are effective to improve hemorrhoids as measured by the standard grading system for hemorrhoids, which grades the condition from grade 1 (least severe) to grade 4 (most severe). In certain embodiments, the formulations disclosed herein are effective to improve the clinical grade of hemorrhoids by at least one grade (e.g., grade 1 to absent), at least two grades (e.g., grade 2 to absent), at least three grades (e.g., grade 3 to absent), or at least four grades (e.g., grade 4 to absent).

In certain embodiments the penetration enhancing agent and/or surfactant ingredient comprises, comprises about, comprises at most, or comprises at most about 20 wt %, 15 wt%, 10 wt% or 5 wt%, of the formulation relative to the rest of the components of the formulation, or any range of values therebetween and comprises, comprises about, comprises at least, or comprises at least about 1 wt%, 0.5 wt%, 0.1 wt%, of the formulation relative to the rest of the components of the formulation, or any range of values therebetween. In certain embodiments of the formulations disclosed herein, suitable penetration enhancing agents and/or surfactants may be selected from the group consisting of polyethylene glycol (PEG), ethoxydiglycol (EDG), diethylene glycol monoethyl ether (“Transcutol”), dimethyl isosorbide (DMI), dimethyl sulfoxide (DMSO), sodium lauryl sulfate (SDS), Polyoxyethylene (20) sorbitan monooleate (Tween 80), and combinations thereof.

In certain embodiments, the composition may comprise a carrier system. The carrier system may comprise humectants, emollients preservatives and solvents and combinations thereof. In certain embodiments, the humectants, emollients preservatives and solvents comprises, comprises about, comprises at most, or comprises at most about 60 wt%, 50 wt%, 40 wt%, 30 wt%, 20 wt%, 10 wt%, of the formulation relative to the rest of the components of the formulation, or any range of values therebetween and comprises, comprises about, comprises at least, or comprises at least about 1 wt%, 2 wt%, 3 wt%, 4 wt%, 5 wt% of the formulation relative to the rest of the components of the formulation, or any range of values therebetween.

Suitable humectants, emollients, preservatives and solvents may be selected from those known in the art, including, e.g., propanediol, hyaluronic acid, sodium hyaluronate, glycerin, propylene glycol, caprylyl glycol, butylene glycol, pentylene glycol, sodium carbomer, horse chestnut extract, aescin, vanillin, xanthan gum, hydroxypropyl methylcellulose, Disodium EDTA, Tetrasodium EDTA, phenoxyethanol, ethylhexylglycerin, benzyl alcohol, diazolidinyl urea, iodopropynyl butylcarbamate DMDM hydantoin, paraben, propyl paraben, methylparaben, polyacrylate crosspolymer, and combinations thereof.

In certain embodiments, the composition may comprise an aqueous component. In certain embodiments, the aqueous component comprises, comprises about, comprises at most, or comprises at most about 90 wt%, 80 wt%, 70 wt%, 60 wt%, 50 wt%, 40 wt%, 30 wt%, 20 wt%, or 10 wt% of the formulation relative to the rest of the components of the formulation, or any range of values therebetween.

In certain embodiments, the formulation for topical treatment of a skin condition comprises the following ingredients and amounts:

TABLE 1

Embodiment 1

| INGREDIENT | REPRESENTATIVE WEIGHT PERCENT |
|---|---|
| P- Synephrine | 1-10%, including, e.g., 1-8%, 1-6%, and 2-4% |
| Ethoxydiglycol | 1-15%, including, e.g., 5-15%, 8-12%, and 9-11% |
| Niacinamide | 1-10%, including, e.g., 4-10%, 6-10%, and 7-9% |
| Glycerin | 1-10%, including, e.g., 1-7%, 2-4%, and 2.5-3.5% |
| Butylenel glycol | 1-15% including, e.g., 5-15%, 8-12%, and 9-11% |
| Benzyl alcohol | 0.1-1%, including, e.g., 0.3-1%, 0.5-1%, and 0.7-0.9% |
| Disodium EDTA | 0.05-0.5%, including, e.g., 0.05-0.4%, 0.5-0.3%, and 0.2-0.3% |
| Ethylhexlglycerin | 0.05-1%, including, e.g.,0.05-0.5%, 0.05-0.3%, and 0.09-0.15% |
| Sodium Carbomer | 0.1-1%, including e.g., 0.3-1%, 0.5-1%, and 0.7-0.9% |
| Water | balance |
| Embodiment 2 | |
| INGREDIENT | REPRESENTATIVE WEIGHT PERCENT |
| Epinephrine | 1-5%, including, e.g., 2-5%, 2.5-4.5%, and 3.5-4.5% |
| Dimethyl Isosorbide | 1-15%, including, e.g., 4-10%, 6-10%, and 7-9% |
| Niacinamide | 1-20% including, e.g., 5-15%, 8-12%, and 9-11% |
| Glycerin | 1-10%, including e.g., 2-9%, 3-7%, and 4-6% |
| Vanillin | 0.5-3%, including, e.g., 0.5-2.5%, 0.5-2%, and 0.5-1.5% |
| Propylene glycol | 1-15% including, e.g., 5-15%, 8-12%, and 9-11% |
| Tetra sodium EDTA | 0.05-0.5% including, e.g., 0.05-0.4%, 0.5-0.3%, and 0.2-0.3% |
| Glycerin | 1-10%, including e.g., 3-9%, 5-8%, and 4-6% |
| Diazolidinyl Urea | 0.05-0.5%, including, e.g., 0.05-0.4%, 0.1-0.3%, and 0.15-0.25% |
| Xanthan Gum | 0.1-1%, including, e.g., 0.1-0.7%, 0.2-0.6%. and 0.3-0.5% |
| Water | balance |
| Embodiment 3 | |
| INGREDIENT | REPRESENTATIVE WEIGHT PERCENT |
| D- Synephrine HCL | 1-20%, including, e.g., 5-18%, 8-15%, and 11-13% |
| Dimethyl isosorbide | 0.1-20%, including, e.g., 5-18%, 10-17%, and 14-16% |
| Vanillin | 0.5-3% including, e.g., 0.5-2.5%, 0.5-2%, and 0.5-1.5% |
| Glycerin | 1-10%, including, e.g., 2-8%, 3-5%, and 1.5-2.5% |
| Pentylene glycol | 1-10%, including, e.g., 3-9%, 4-9%, and 5-8% |
| Benzyl alcohol | 0.1-1%, including, e.g., 0.3-0.9%, 0.5-0.9%, and 0.6-0.8% |
| Disodium EDTA | 0.05-0.5% including, e.g., 0.05-0.4%, 0.5-0.3%, and 0.2-0.3% |
| Sodium hyaluronate | 0.1-1%, including, e.g., 0.3-0.9%, 0.4-0.8%, and 0.5-0.7% |
| DMDM hydantoin | 0.05-1%, including, e.g., 0.05-0.6%, 0.05-0.5%, and 0.1-0.3% |
| Water | balance |
| Embodiment 4 | |
| INGREDIENT | REPRESENTATIVE WEIGHT PERCENT |
| L- Synephrine | 1-10%, including, e.g., 3-9%, 4-8%, and 5-7% |
| Dimethyl isosorbide | 1-10%, including, e.g., 3-9%, 4-8%, and 5-7% |
| Niacinamide | 1-10%, including, e.g., 4-10%, 6-10%, and 7-9% |
| Glycerin | 0.5-10% including, e.g., 0.5-8%, 0.5-5%, and 0.5-1.5% |
| Butylene glycol | 1-10% including, e.g., 4-10%, 6-10%, and 7-9% |
| Disodium EDTA | 0.01-0.1%, including, e.g., 0.02-0.08%, 0.03-0.07%, and 0.04-0.06% |
| Hyaluronic acid | 0.1-1% including, e.g., 0.3-0.9%, 0.5-0.9%, and 0.6-0.8% |
| Propylparaben | 0.2-1%, including, e.g., 0.2-0.8%, 0.2-0.6%, and 0.2-0.4% |
| Methylparaben | 0.2-1%, including, e.g.,0.2-0.8%, 0.3-0.6%, 0.4-0.6% |
| Water | balance |
| Embodiment 5 | |
| INGREDIENT | REPRESENTATIVE WEIGHT PERCENT |
| Synephrine HCL | 1-10% including, e.g., 4-10%, 6-10%, and 7-9% |
| Dimethyl isosorbide | 1-20% including, e.g., 5-15%, 8-12%, and 9-11% |
| Niacinamide | 1-10% including, e.g., 4-10%, 6-10%, and 7-9% |
| Glycerin | 1-10% including, e.g., 2-8%, 3-5%, and 1.5-2.5% |
| Propane diol | 1-15% including, e.g., 5-15%, 8-12%, and 9-11% |
| Benzyl alcohol | 0.1-1%, including, e.g., 0.1-0.8%, 0.2-0.7%, and 0.4-0.6% |
| Disodium EDTA | 0.01-0.1% including, e.g., 0.02-0.08%, 0.03-0.07%, and 0.04-0.06% |
| Hydroxylproply methycellulose | 0.1-1% including, e.g., 0.02-0.08%, 0.03-0.07%, and 0.04-0.06% |
| Phenoxyethanol | 0.2-1% including, e.g., 0.3-0.9%, 0.4-0.8%, and 0.5-0.7% |
| Water | balance |
| Embodiment 6 | |
| INGREDIENT | REPRESENTATIVE WEIGHT PERCENT |
| Synephrine HCL | 0.5-10%, including e.g., 3-9%, 5-8%, and 4-6% |
| Niacinamide | 1-10%, including, e.g., 2-9%, 2-6%, and 3-5% |
| Glycerin | 1-10%, including, e.g., 2-8%, 2-6%, and 3-5% |
| Ethoxydiglycol | 1-15%, including, e.g., 4-10%, 6-10%, and 7-9% |
| Propanediol | 1-15%, including, e.g., 5-15%, 8-12%, and 9-11% |
| Benzyl alcohol | 0.1-1% including, e.g., 0.1-0.8%, 0.2-0.7%, and 0.4-0.6% |
| Disodium EDTA | 0.05-0.5%, including, e.g., 0.05-0.4%, 0.5-0.3%, and 0.2-0.3% |
| Polyacrylate Crosspolymer-6 | 0.1-1%, including, e.g., 0.3-0.9%, 0.4-0.8%, and 0.5-0.7% |
| Caprylyl Glycol | 0.1-1%, including, e.g., 0.2-0.8%, 0.2-0.6%, and 0.2-0.4% |
| Water | balance |
| Embodiment 7 | |
| INGREDIENT | REPRESENTATIVE WEIGHT PERCENT |
| P- Synephrine | 1-15%, including, e.g., 6-14%, 8-13%, and 10-12% |
| Ethoxydiglycol | 1-15%, including, e.g., 5-14%, 7-13%, and 9-11% |
| Niacinamide | 1-15%, including, e.g., 4-14%, 6-12%, and 8-10% |
| Glycerin | 1-10%, including, e.g., 1-8%, 1-6%, and 2-5% |
| Butylenel glycol | 1-15%, including, e.g., 6-14%, 8-12%, and 9-11% |
| Benzyl alcohol | 0.1-1%, including, e.g., 0.3-0.9%, 0.5-0.9%, and 0.7-0.9% |
| Disodium EDTA | 0.05-0.5% including, e.g., 0.05-0.4%, 0.5-0.3%, and 0.2-0.3% |
| DMDM Hydantoin | 0.05-1% including, e.g., 0.05-0.07%, 0.05-0.04% and 0.1-0.3% |
| Water | balance |
| Embodiment 8 | |
| INGREDIENT | REPRESENTATIVE WEIGHT PERCENT |
| Epinephrine | 1-5%, including, e.g., 2-5%, 3-5%, and 3.5-4.5% |
| Dimethyl Isosorbide | 1-15%, including, e.g., 4-14%, 6-12%, and 7-9% |
| Niacinamide | 1-15% including, e.g., 6-14%, 8-12%, and 9-11% |
| Glycerin | 1-10% including e.g., 3-9%, 5-8%, and 4-6% |
| Vanillin | 0.5-3%, including, e.g., 0.5-2.5%, 0.5-2%, and 0.5-1.5% |
| Propylene glycol | 1-15% including, e.g., 6-14%, 8-12%, and 9-11% |
| Tetra sodium EDTA | 0.05-0.15%, including, e.g., 0.06-0.15%, 0.07-0.12%, and 0.09-0.1% |
| Glycerin | 1-10%, including e.g., 3-9%, 5-8%, and 4-6% |
| DMDM Hydantoin | 0.05-1%, including, e.g., 0.05-0.07%, 0.05-0.04% and 0.1-0.3% |
| Xanthan Gum | 0.1-1%, including, e.g., 0.1-0.7%, 0.2-0.6%. and 0.3-0.5% |
| Water | balance |
| Embodiment 9 | |
| INGREDIENT | REPRESENTATIVE WEIGHT PERCENT |
| D- Synephrine HCL | 1-20%, including, e.g., 5-15%, 8-12%, and 9-11% |
| Dimethyl isosorbide | 0.1-20%, including, e.g., 4-15%, 6-13%, and 7-9% |
| Vanillin | 0.5-3% including, e.g., 0.5-2.5%, 0.5-2%, and 0.5-1.5% |
| Glycerin | 1-10%, including, e.g., 1-8%, 2-6%, and 2-5% |
| Pentylene glycol | 1-10%, including, e.g., 3-9%, 4-9%, and 5-8% |
| Benzyl alcohol | 0.1-1%, including, e.g., 0.3-1%, 0.5-0.9%, 0.7-0.8% |
| Disodium EDTA | 0.01-0.5% including, e.g., 0.03-0.4%, 0.04-0.2%, and 0.05-0.1% |
| Sodium hyaluronate | 0.1-1%, including, e.g., 0.3-0.9%, 0.4-0.8%, and 0.5-0.7% |
| Water | balance |
| Embodiment 10 | |
| INGREDIENT | REPRESENTATIVE WEIGHT PERCENT |
| Synephrine HCL | 1-15%, including, e.g., 6-14%, 8-13%, and 10-12% |
| Dimethyl isosorbide | 1-20%, including, e.g., 5-15%, 7-12%, and 7-9% |
| Niacinamide | 1-15% including, e.g., 4-14%, 6-12%, and 8-10% |
| Glycerin | 1-10%, including e.g., 2-9%, 3-7%, and 3-5% |
| Propane diol | 1-15%, including, e.g., 5-15%, 8-12%, and 9-11% |
| Disodium EDTA | 0.01-0.5%, including, e.g., 0.03-0.4%, 0.04-0.2%, and 0.05-0.1% |
| Hydroxylproply methycellulose | 0.01-1%, including, e.g., 0.02-0.08%, 0.03-0.07%, and 0.04-0.06% |
| Phenoxyethanol | 0.2-1%, including, e.g., 0.3-0.9%, 0.5-0.9%, and 0.6-0.9% |
| Water | balance |
| Embodiment 11 | |
| INGREDIENT | REPRESENTATIVE WEIGHT PERCENT |
| Synephrine HCL | 0.5-10%, including, e.g., 3-9%, 5-8%, and 4-6% |
| Ethoxydiglycol | 1-10%, including, e.g., 4-10%, 6-10%, and 7-9% |
| Niacinamide | 1-10%, including, e.g., 2-9%, 2-6%, and 3-5% |
| Glycerin | 1-10%, including, e.g., 2-8%, 2-6%, and 3-5% |
| Propanediol | 1-15%, including, e.g., 5-15%, 8-12%, and 9-11% |
| Phenoxyethanol | 0.1-1% including, e.g., 0.3-0.9%, 0.4-0.8%, and 0.5-0.7% |
| Disodium EDTA | 0.05-0.1%, including, e.g., 0.05-0.09%, 0.06-0.09%, and 0.07-0.09% |
| Polyacrylate Crosspolymer-6 | 0.1-1%, including, e.g., 0.3-0.9%, 0.4-0.8%, and 0.5-0.7% |
| Caprylyl Glycol | 0.1-1%, including, e.g., 0.2-0.8%, 0.2-0.6%, and 0.2-0.4% |
| Water | balance |

Embodiment 12

| | |
|---|---|
| INGREDIENT | WEIGHT PERCENT |
| Disodium EDTA | 0.01-0.5%, including, e.g., 0.03-0.4%, 0.04-0.2%, and 0.05-0.1% |
| Niacinamide | 1-10%, including, e.g., 4-10%, 6-10%, and 7-9% |
| Synephrine HCL | 0.5-10%, including e.g., 3-9%, 5-8%, and 4-6% |
| Dimethyl isosorbide | 1-20% including, e.g., 5-15%, 8-12%, and 9-11% |
| Propanediol | 1-15% including, e.g., 5-15%, 8-12%, and 9-11% |
| Glycerin | 1-10%, including, e.g., 1-7%, 2-4%, and 2.5-3.5% |
| Phenoyethanol | 0.2-1% including, e.g., 0.3-0.9%, 0.4-0.8%, and 0.5-0.7% |
| Caprylyl Glycol | 0.05-0.5% including, e.g., 0.05-0.4%, 0.05-0.3%, and 0.05-0.2% |
| Hyaluronic Acid | 0.1-1% including, e.g., 0.3-0.9%, 0.5-0.9%, and 0.7-0.9% |
| Water | balance |

According to certain embodiments, the topical formulation can be in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter.

Topical formulations include those in which any other active ingredient(s) is (are) dissolved or dispersed in a dermatological vehicle known in the art (e.g., aqueous or nonaqueous gels, ointments, water-in-oil or oil-in-water emulsions). Constituents of such vehicles can comprise water, aqueous buffer solutions, non-aqueous solvents (e.g., ethanol, isopropanol, benzyl alcohol, 2-(2-ethoxyethoxy)ethanol, propylene glycol, propylene glycol monolaurate, glycofurol or glycerol), oils (e.g., a mineral oil such as a liquid paraffin, natural or synthetic triglycerides, or silicone oils such as dimethicone). Depending, inter alia, upon the nature of the formulation as well as its intended use and site of application, the dermatological vehicle employed can contain one or more components (e.g., when the formulation is an aqueous gel, components in addition to water) selected from the following list: a solubilizing agent or solvent (e.g., a β-cyclodextrin, such as bydroxypropyl, or an alcohol or polyol such as ethanol, propylene glycol or glycerol); a thickening agent (e.g., hydroxyethylceliulose, hydroxypropylcellulose, carboxymethylcellulose or carbomer); a gelling agent (e.g., a polyoxyethylene-polyoxypropylene copolymer); a preservative (e.g., benzyl alcohol, benzalkonium chloride, chlorhexidine, chlorbutol, a benzoate, potassium sorbate or EDTA or salt thereof); and pH buffering agent(s) (e.g., a mixture of dihydrogen phosphate and hydrogen phosphate salts, or a mixture of citric acid and a hydrogen phosphate salt).

A pharmaceutically acceptable carrier can also be incorporated in the formulation of the present disclosure and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted, interaction with other components of the formulation. “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

According to certain embodiments, the formulations disclosed herein may comprise a film former. A film former, when it dries, forms a protective film over the site of application. The film former inhibits removal of the active ingredient and keeps it in contact with the site being treated. An example of a film former is Flexible Collodion, US P. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which particles, including the active agent, are present in a water or alcohol base. Lotions are usually suspensions of solids, and in some embodiments, comprise a liquid oily emulsion of the oil-in-water type. In some embodiments, lotion formulations are used herein for treating large body areas, because of the ease of applying a more fluid composition. It is generally necessary that the insoluble matter in a lotion be finely-divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize, or preserve the solute. Common examples of solvents used in preparing solutions are ethanol, water, propylene glycol or any other acceptable vehicles. As is well known, gels are semisolid, suspension-type systems. Single-phase gels contain organic macromolecules distributed substantially uniformly throughout the carrier liquid, which is typically aqueous, but also, in some embodiments, contains an alcohol, and, optionally, an oil. In some embodiments, “organic macromolecules,” are used, i.e., gelling agents, are cross-linked acrylic acid polymers such as the “carbomer” family of polymers, e.g., carboxypolyalkylenes that can be obtained commercially under the Carbopol trademark. In other embodiments, hydrophilic polymers are used such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthaiate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Enhancers are those lipophilic co-enhancers typically referred to as “plasticizing” enhancers, i.e., enhancers that have a molecular weight in the range of about 150 to 1000, an aqueous solubility of less than about 1 wt.%, in some embodiments less than about 0.5 wt.%, and most in some embodiments less than about 0.2 wt.%. The Hildebrand solubility parameter 6 of plasticizing enhancers is in the range of about 2.5 to about 10, in some embodiments in the range of about 5 to about 10. In some embodiments lipophilic enhancers are fatty esters, fatty alcohols, and fatty ethers. Examples of specific fatty acid esters include methyl laurate, ethyl oleate, propylene glycol nionolaurace, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, in some embodiments, a C₂-C₄ alkane diol or triol, are substituted with one or two fatty ether substituents. Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995)(incorporated herein by reference).

Various other additives can be included in the compositions of the present disclosure in addition to those identified above. These include, but are not limited to, antioxidants, astringents, perfumes, preservatives, emollients, pigments, dyes, humectants, propeliants, and sunscreen agents, as well as other classes of materials whose presence can be pharmaceutically or otherwise desirable. Typical examples of optional additives for inclusion in the formulations of the disclosure are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), α-tocopherol (Vitamin E), β-tocopherol, gamma-tocopherol, delta-tocopherol, epsilon-tocopherol, zeta-tocopherol, Z GAMMA-tocopherol, eta-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), α-tocopherol (Vitamin E), beta-tocopherol, gamma-tocopherol, delta-tocopherol, epsilon-tocopherol, zeta-tocopherol, zeta 2-tocopherol, eta-tocopherol, and retinol (Vitamin A), and/or pharmaceutically acceptable salts, esters, amides, or other derivatives thereof. In some embodiments the tocopherol compound is α-tocopherol. Additional agents include those that are capable of improving oxygen supply in skin tissue, as described, for example, in Gross, et al, WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycmnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene. See, Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the disclosure. Such healing materials can include nutrients, minerals, vitamins, electrolytes, enzymes, herbs, plant extracts, glandular or animal extracts, or safe therapeutic agents that can be added to the formulation to facilitate the healing of dermal disorders. The amounts of these various additives are those conventionally used in the cosmetics field, and range, for example, from about 0.01% to about 20% of the total weight of the topical formulation.

The formulations of the disclosure can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds.

The formulations can also contain irritation-mitigating additives to minimize or eliminate the possibility of skin irritation or skin damage resulting from the chemical entity to be administered, or other components of the composition. Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt.%, more typically not more than about 5 wt.%, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites. Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The pharmaceutical compositions of the disclosure comprise one or more active ingredients, e.g. therapeutic agents, in admixture with one or more pharmaceutically-acceptable diluents or carriers and, optionally, one or more other compounds, drugs, ingredients and/or materials. Regardless of the route of administration selected, the agents/compounds of the present disclosure are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer’s injection, dextrose injection, dextrose and sodium chloride injection, lactated Ringer’s injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the disclosure must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the disclosure may, optionally, contain additional ingredients and/or materials commonly used in pharmaceutical and/or over-the-counter compositions. These ingredients and materials are well known in the art and non-limiting examples include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

### Methods of Making the Formulations

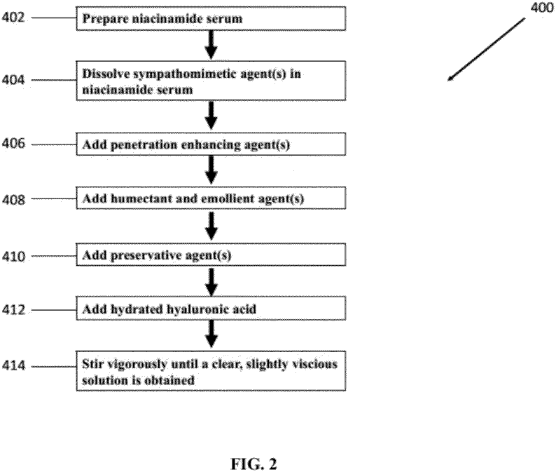

According to some aspects, the present disclosure provides methods of manufacturing the formulations disclosed herein. For example, FIG. 2 is a flow chart of an embodied process 400 for manufacturing formulations for topical application of sympathomimetics. A niacinamide serum is provided at a state 402. A sympathomimetic active agent is then provided with mixing at state 404. Following this penetration enhancing agents including surfactants are provided with mixing at state 406. Then humectants and emollient agents are provided with mixing at state 408, following by preservative agents at state 410. In state 412 hydrated hyaluronic is provided with mixing. In certain embodiments, 412 may be achieved by first dissolving hyaluronic acid in a portion of solvent reserved from state 408, which is then added to state 410 to reach state 412. Finally in state 414 the formulation is mixed by stirring for 1-5 hours until a clearly, slightly viscous solution is obtained.

According to certain aspects, the present disclosure provides methods of manufacturing the formulations disclosed herein using the following materials, equipment, and procedures.

According to certain such embodiments, the procedure for making a 500 g batch of formulation is as follows: (1) Place a beaker on a hot plate under an overhead stirrer and add water, then adjust water temperature to 20-25° C.; (2) Turn overhead stirrer on and set to 300 RPM; (3) Add a chelator and stir until fully dissolved; (4) Add niacinamide to the product of step (3) and stir until fully dissolved; (5) Add sympathomimetic agent to the product of step (4) and stir until fully dissolved; (6) Add penetration enhancing agent to the product of step (5); (7) add humectant and emollients to the product of step (6); (8) Add preservative agents to the product of step (7); (9) In a separate beaker, dissolve hyaluronic acid and stir until thoroughly mixed; (10) Add the product of step 9 to the product of step (8) with stirring; (11) Test and adjust (if necessary) the pH of the product of step 10 to the approximate range of 5-6. (12) Stir the product of step (11) until the mixture has formed a transparent solution (approx. 2-3 hours).

### Methods of Treatment

According to some aspects, the present disclosure provides methods of treatment of skin conditions using a vasoconstrictor. In some aspects, the present disclosure refers to the surprising and unexpected finding that using sympathomimetic compounds (alone or in combination) together with a delivery system comprising permeation enhancers is able to penetrate the skin to effect local vasoconstriction when applied to the skin of a subject. See, e.g., Examples 1-7, below. As used herein, a vascular condition is a medical condition involving skin vasculature that may be treated using the formulations disclosed herein. Such vascular conditions include, but are not limited to hemorrhoids, spider veins, varicose veins, rosacea, and periorbital edema.

In certain embodiments, the present disclosure provides a method for the treatment or lessening the severity of vascular conditions, such as certain skin/membrane conditions (e.g., hemorrhoids, spider veins, varicose veins, rosacea, and periorbital edema) comprising administering an effective amount of a formulation disclosed herein to a subject in need thereof. In certain embodiments of the present disclosure an “effective amount” of the compound or pharmaceutically acceptable composition is that amount effective for treating or lessening the severity of a vascular condition, such as certain skin/membrane conditions, including hemorrhoids, spider veins, varicose veins, rosacea, and periorbital edema. The formulas and compositions according to the methods disclosed herein may be administered using any amount and any topical form of administration effective for treating or lessening the severity of a vascular condition, such as certain skin/membrane conditions, including hemorrhoids, spider veins, varicose veins, rosacea, and periorbital edema. The exact amount required will vary from subject to subject, depending on the species, age, and general condition of the subject, the severity of the condition, the particular agent, its mode of administration, and the like. The formulas disclosed herein are, in some embodiments, formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions disclosed herein will be decided by the attending physician within the scope of sound medical judgment. The specific effective dose level for any particular subject or organism will depend upon a variety of factors including the disorder being treated and the severity of the disorder; the activity of the specific compound employed; the specific composition employed; the age, body weight, general health, sex and diet of the subject; the time of administration, topical method of administration; the duration of the treatment; drugs used in combination or coincidental with the specific compound employed, and like factors well known in the medical arts. The term “subject”, as used herein, means an animal, preferably a mammal, and most preferably a human.

Dosage forms for topical administration are disclosed above. Ophthalmic formulation, ear drops, and eye drops are also contemplated as being within the scope of this disclosure. Additionally, the present disclosure provides the use of skin patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In certain embodiments, the formulations disclosed herein are applied once daily, twice daily, three times daily, or more. In certain embodiments, the formulations disclosed herein are applied for more than 5 days, more than 10 days, more than 20 days, or more than 30 days. In certain embodiments, the formulations disclosed herein are applied twice daily for about 20 days (e.g., for treatment of hemorrhoids). According to certain embodiments, the formulations disclosed herein are applied once daily for about 30 days (e.g., for treatment of spider veins). According to certain embodiments, the formulations disclosed herein are applied once daily for about 35 days (e.g., for treatment of varicose veins). According to certain embodiments, the formulations disclosed herein are applied serially (e.g., as needed for treatment of rosacea or periorbital edema).

While certain embodiments have been described, these embodiments have been presented by way of example only, and are not intended to limit the scope of the disclosure. Indeed, the novel methods and systems described herein may be embodied in a variety of other forms, Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics or groups described in conjunction, with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover while operations may be depicted in the drawings or described it he specification in particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in certain embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “‘substantially” may refer to an amount that is within less than 10% of, within less than 5% of, within less than 1% of, within less than 0.1% of, and within less than 0.01% of the stated amount, depending on the desired function or desired result.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed m the claims and not limited to the examples described in the present specification or during the prosecution of the application, which examples are to be construed as non-exclusive.

The headings provided herein, if any, are for convenience only and do not necessarily affect the scope or meaning of the devices and methods disclosed herein.

### Examples

TABLE 2

EXAMPLE 1
Representative process example for production of a 500 g batch size of the following representative formula

| | |
|---|---|
| INGREDIENT | WEIGHT PERCENT |
| Disodium EDTA | 0.05 |
| Niacinamide | 8 |
| Synephrine HCL | 5 |
| Dimethyl isosorbide | 10 |
| Propanediol | 10 |
| Glycerin | 3 |
| Phenoyethanol | 0.6 |
| Caprylyl Glycol | 0.1 |
| Hyaluronic Acid | 0.8 |
| Water | balance |

The materials comprise the following: Disodium EDTA / 250 mg; Demineralized H₂O / 312 ml; Hyaluronic Acid / 4 grams; Dimethyl Isosorbide / 50 grams; Propanediol / 50 grams; Phenoxyethanol / 3 grams; Caprylyl Glycol / 500 mg; Glycerin / 15 grams; Synephrine HCL / 25 grams; Niacinamide / 40 grams; 1000 ml Beaker; 100 ml weighing dish; 100 ml Beaker; Thermometer; Glass stir rod; Pipettes. The equipment comprises the following: Overhead stirring apparatus with propeller attachment; scale; hot plate; pH meter.

The procedure for making a 500 g batch of formulation is as follows: (1) Place a 1000 ml beaker on a hot plate under an overhead stirrer and add 312 ml water, then adjust water temperature to 20-25° C.; (2) Turn overhead stirrer on and set to 300 RPM; (3) Add disodium EDTA and stir until fully dissolved; (4) Add Niacinamide to the product of step (3) and stir until fully dissolved; (5) Add synephrine HCl to the product of step (4) and stir until fully dissolved; (6) Add dimethyl isosorbide and glycerin to the product of step (5); (7) Add 25 grams of propanediol to the product of step (6); (8) Add phenoxyethanol and caprylyl glycol to the product of step (7); (9) In a 100 mL beaker, mix hyaluronic acid with 25 g of propanediol and stir until thoroughly mixed; (10) Add the product of step 9 to the product of step (8) with stirring at 500 RPM; (11) Test and adjust (if necessary) the pH of the product of step 10 to the approximate range of 5-6. (12) Stir the product of step (11) until the mixture has formed a transparent solution (approx. 2-3 hours).

TABLE 3

EXAMPLE 2
The following exemplary formula was manufactured for topical treatment of hemorrhoids. The ingredients and weight percent ranges are as follows

| | |
|---|---|
| INGREDIENT | WEIGHT PERCENT |
| P- Synephrine HCl | 10 |
| Ethoxydiglycol | 10 |
| Niacinamide | 8 |
| Glycerin | 3 |
| Butylene glycol | 10 |
| Benzyl alcohol | 0.8 |
| Disodium EDTA | 0.1 |
| DMDM Hydantoin | 0.2 |
| Water | balance |

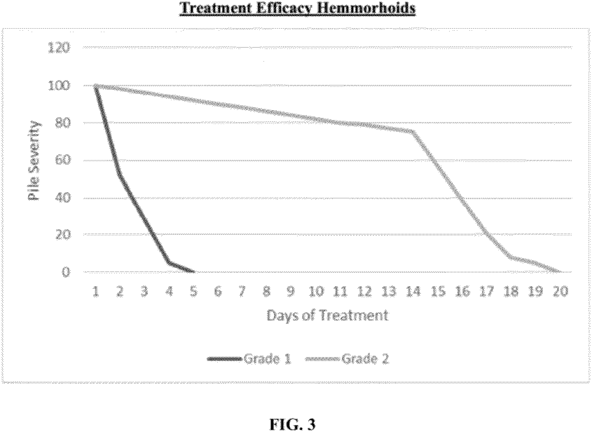

FIG. 3 is a chart showing the results of an experiment wherein hemorrhoids were treated using the formulation of Example 2. At the start of the experiment the hemorrhoids were assessed using the Banov grading scale which classifies hemorrhoids by their degree of prolapse into the anal canal. The test subject was diagnosed as having two prolapsed grade 1 piles on the anterior anus and a large thrombosed pile on the posterior side that was also bleeding. The formulation of Example 1 was topically applied to all three piles twice daily. After approximately 4 treatments over 2 days the prolapsed grade 1 piles were no longer visible, and the test subject reported a reduction in pain levels. The larger thrombosed grade 2 pile exhibited an approximately 25% reduction after 14 days and 24 topical applications. Between days 14 and 20 that pile then bifurcated into two smaller piles which finally disappeared from external view after approximately 20 days and 40 applications. The test subject reported a reduction in pain levels and did not observe further bleeding. That test subject also reported no return or reappearance of hemorrhoids in the affected area for at least 20 days following treatment. FIG. 3 demonstrates that the formulation of Example 2 provided improved hemorrhoid treatment relative to existing topical treatment options. Even when the targeted vasculature is located at or near the surface of the skin, penetration of sympathomimetic active ingredients around and underneath that vasculature provides considerable improvement in the speed and effect of drug action.

TABLE 4

EXAMPLE 3
EXAMPLE 3. The following exemplary formula was manufactured for topical treatment of spider veins. The ingredients and weight percent ranges are as follows

| | |
|---|---|
| INGREDIENT | WEIGHT PERCENT |
| Epinephrine | 4 |
| Dimethyl Isosorbide | 8 |
| Niacinamide | 10 |
| Glycerin | 5 |
| Vanillin | 1 |
| Propylene glycol | 10 |
| Tetra sodium EDTA | 0.1 |
| DMDM Hydantoin | 0.2 |
| Xanthan Gum | 0.4 |
| Water | balance |

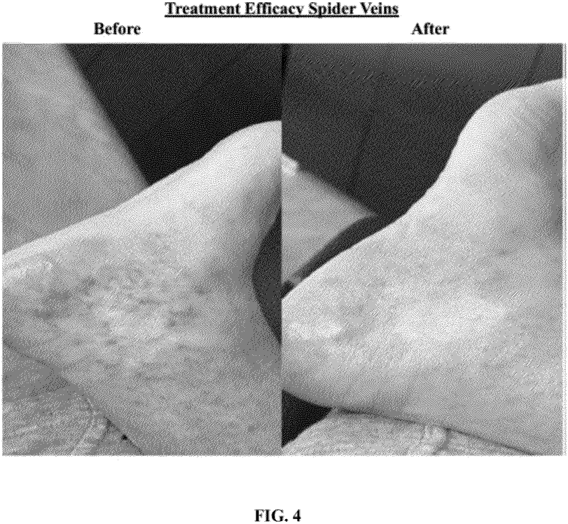

Patient presented with spider veins on the lateral tarsal (ankle) area of the left leg. The micrographs in FIG. 4 show the results of an experiment wherein spider veins were treated using the formulation in Example 3. At the start of the experiment the veins were assessed using the CEAP classification system. The test subject was diagnosed as having a CEAP clinical score of C1, or telangectasic, thread veins, spider veins, and/or broken veins. The formulation of Example 2 was topically applied (approximately 0.5 mls)on and around the affected vasculature once daily. After 45 minutes the spider veins had mostly disappeared (after). According to the clinical etiological anatomical pathophysiological (CEAP) classification this patient went from a CEAP score of 1 to a CEAP score of 0 with no visual evidence of chronic venous disease (CVD) in that area. After approximately 30 sequential days of treatment venous pathology was almost eliminated with only a few sites of pathology remaining detectable. FIG. 4 demonstrates that the formulation of Example 3 enables an effective, and convenient topical treatment of spider veins that was not previously available.

TABLE 5

EXAMPLE 4
The following exemplary formula was manufactured for topical treatment of varicose veins. The ingredients and weight percent ranges are as follows

| | |
|---|---|
| INGREDIENT | WEIGHT PERCENT |
| D- Synephrine HCL | 12 |
| Dimethyl isosorbide | 15 |
| Vanillin | 1 |
| Glycerin | 2 |
| Pentylene glycol | 6 |
| Benzyl alcohol | 0.7 |
| Disodium EDTA | 0.1 |
| Sodium hyaluronate | 0.6 |
| Water | balance |

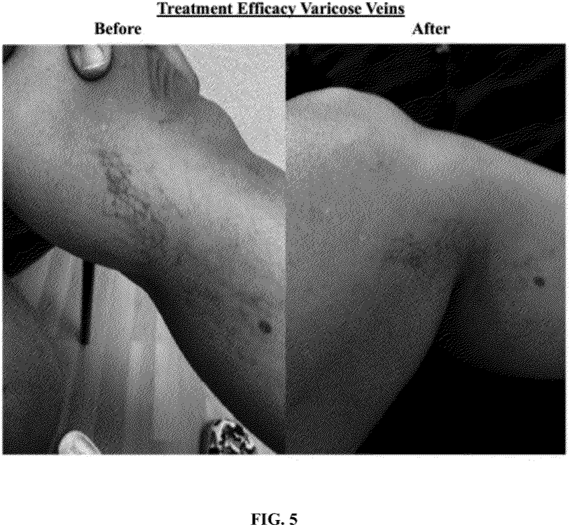

Patient presented with visible varicose veins on the medial left patella (knee) spanning a large area. The micrographs in FIG. 5 show the results of an experiment wherein varicose veins located on the medical surface of the knee were treated using the formulation in Example 4. At the start of the experiment the veins were assessed using the CEAP classification system. The test subject was diagnosed as having a CEAP clinical score of C2S, or varicose veins with symptoms (before). The formulation of Example 4 was topically applied (approximately 0.75 mL) on and around affected vasculature twice daily. After 2 days, the varicose veins disappeared in much of the area (CEAP score of 0) and one area reduces to a CEAP score of 1 (spider veins). After approximately 35 sequential days of treatment venous pathology was significantly reduced and significant cosmetic benefit was observed. FIG. 5 demonstrates that the formulation of Example 4 enables an effective, and convenient topical treatment of varicose veins relative to that was not previously available.

TABLE 6

EXAMPLE 5
Composition for topical treatment of rosacea by ingredient and weight percent range

| | |
|---|---|
| INGREDIENT | WEIGHT PERCENT |
| L- Synephrine | 6 |
| Dimethyl isosorbide | 6 |
| Niacinamide | 8 |
| Glycerin | 1 |
| Butylene glycol | 8 |
| Disodium EDTA | 0.05 |
| Hyaluronic acid | 0.7 |
| Propylparaben | 0.3 |
| Methylparaben | 0.5 |
| Water | balance |

The composition will be applied 1-5 times daily according to patient needs for 1-30 days to subjects presenting with pre-rosacea, stage I rosacea, stage II rosacea, and stage III rosacea. The results will show a significant reduction of the clinical grade of rosacea after application to the affected area.

TABLE 7

EXAMPLE 6
Composition for topical treatment of periorbital edema by ingredient and weight percent range

| | |
|---|---|
| INGREDIENT | WEIGHT PERCENT |
| Synephrine HCL | 8 |
| Dimethyl isosorbide | 10 |
| Niacinamide | 8 |
| Glycerin | 2 |
| Propane diol | 10 |
| Disodium EDTA | 0.05 |
| Hydroxylproply methycellulose | 0.5 |
| Phenoxyethanol | 0.6 |
| Caprylyl glycol | 0.4 |
| Water | balance |

The composition will be applied 1-5 times daily according to patient needs for 1-30 days to subjects presenting with periorbital edema. The results will show a significant reduction of the periorbital edema after application to the affected area.

TABLE 8

EXAMPLE 7
Composition for topical treatment of superficial vascular conditions involving vessel wall ruptures, valve damage, twisting and enlargement by ingredient and weight percent range

| | |
|---|---|
| INGREDIENT | WEIGHT PERCENT |
| Synephrine HCL | 5 |
| Ethoxydiglycol | 8 |
| Niacinamide | 4 |
| Glycerin | 4 |
| Propanediol | 10 |
| Disodium EDTA | 0.1 |
| Polyacrylate Crosspolymer-6 | 0.6 |
| Caprylyl Glycol | 0.3 |
| Phenoxyethanol | 0.9 |
| Water | balance |

The composition will be applied 1-5 times daily according to patient needs for 1-30 days to subjects presenting with superficial vascular conditions. The results will show a significant reduction of the clinical grade of superficial vascular conditions after application to the affected area.

TABLE 9

EXAMPLE 8
The following exemplary formula was manufactured for topical treatment of hemorrhoids. The ingredients and weight percent ranges are as follows

| | |
|---|---|
| INGREDIENT | WEIGHT PERCENT |
| P-Synephrine HCl | 15 |
| Dimethyl Isosorbide | 15 |
| Propanediol | 10 |
| Niacinamide | 8 |
| Caprylyl Glycol | 0.3 |
| Phenoxyethanol | 0.7 |
| Disodium EDTA | 0.05 |
| Hyaluronic acid | 0.5 |
| Water | balance |

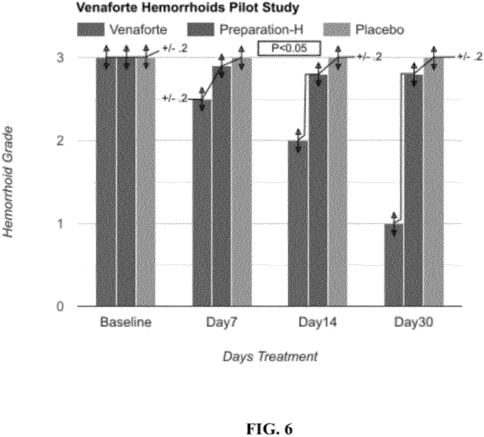

FIG. 6 is a chart showing the results of an experiment wherein hemorrhoids were treated using the formulation of Example 8 in comparison to PREPARATION-H™ and Placebo. At the start of the experiment the hemorrhoids were assessed using the Banov grading scale which classifies hemorrhoids by their degree of prolapse into the anal canal. The test subjects were diagnosed as having an average of grade 2 hemorrhoids at day 1. For group A, 4 patients used the formulation of Example 8. The formulation was topically applied twice daily by direct application using an applicator or by manual application with fingers. The formulation was applied twice daily for 30 days. For group B, 4 patients used PREPARATION-H™ according to the manufacturer’s suggested use, which includes both application with an applicator or by manual application with fingers up to four times a day. For group C, 4 patients used a placebo twice a day for 30 days. Patients were blinded to the study by labeling scheme of each product.

Areas were assessed by using self-palpitating, self-assessment, or by practitioner assessment to quantify number and size of hemorrhoids at 0, 3, 7, 14, and 30 days.

The patients in group A being treated with the formulation of Example 8 (Venaforte), had a reduction in visibility and size of hemorrhoids. This group had hemorrhoids starting at a grade 3 (prolapse) and after 30 days showed a reduction in grade to grade 1 (internal hemorrhoids which do not prolapse). Patients in group A also reported a reduction in discomfort, from the increase in microcirculation helping excess localized fluid retention in the areas of the piles. Group B had no change in hemorrhoid size as is depicted in FIG. 6. Group C had no change in hemorrhoid size as is depicted in FIG. 6.

TABLE 10

EXAMPLE 9
The following exemplary formula was manufactured for topical treatment of varicose veins and spider veins. The ingredients and weight percent ranges are as follows

| | |
|---|---|
| INGREDIENT | WEIGHT PERCENT |
| P-Synephrine HCl | 15 |
| Dimethyl Isosorbide | 15 |
| Propanediol | 10 |
| Niacinamide | 8 |
| Caprylyl Glycol | 0.3 |
| Phenoxyethanol | 0.7 |
| Disodium EDTA | 0.05 |
| Hyaluronic acid | 0.5 |
| Water | balance |

Patients with varicose veins and spider veins located on the interior ankle, side of calve, or upper thighs were treated using the formulation in Example 9 in comparison to PREPARATION-H® and placebo. The size of affected areas were assessed by manual measurement using a ruler to measure dimensions of visible veins. Changes over time were quantified as a percentage of area over which varicose veins were visible. The formulation of Example 9 was topically applied to 4 patients in group A, with an applicator or by manual application (approximately 1 mL) on and around affected vasculature twice daily for 30 days. Group B was 4 patients and these patients used PREPARATION-HPatent® applied by hand (approximately 1 ml) and rubbed onto target area twice daily for 30 days. Group C was 4 patients using a placebo twice daily for 30 days.

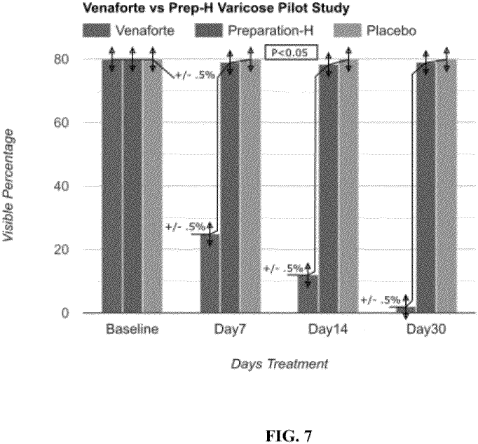

Patients were blinded to the product using a labelling scheme. For group A, as is depicted in FIG. 7, the patients using the formulation of Example 9 showed a reduction of ~80% in visibility and size of the spider veins and varicose veins (veins ranged in size from 2 mm - 4 mm). Patients in group A also noticed a reduction in discomfort from the increase in microcirculation helping reduce the excess localized fluid retention in the area of the veins. The patients in group B, as is depicted in FIG. 7, showed no change in visibility of varicose veins or spider veins. The patients in group C also showed no change in visibility of varicose veins or spider veins.

TABLE 11

EXAMPLE 10
The following exemplary formula was manufactured for topical treatment of varicose veins and spider veins. The ingredients and weight percent ranges are as follows

| | |
|---|---|
| INGREDIENT | WEIGHT PERCENT |
| P-Synephrine HCl | 15 |
| Dimethyl Isosorbide | 15 |
| Propanediol | 10 |
| Niacinamide | 8 |
| Caprylyl Glycol | 0.3 |
| Phenoxyethanol | 0.7 |
| Disodium EDTA | 0.05 |
| Hyaluronic acid | 0.5 |
| Water | balance |

Patients with varicose veins and spider veins located on the interior ankle, side of calve, or upper thighs were treated using the formulation in Example 10 in comparison to PHARMAPULSE™ and placebo. The size of affected areas were assessed by manual measurement using a ruler to measure dimensions of visible veins. Changes over time were quantified as a percentage of area over which varicose veins were visible. The formulation of Example 10 was topically applied to 4 patients in group A, with an applicator or by manual application (approximately 1 mL) on and around affected vasculature twice daily for 30 days. Group B was 4 patients and these patients used PHARMAPULSE™ applied according to manufacturer’s suggested usage for 30 days. Group C was 4 patients using a placebo twice daily for 30 days.

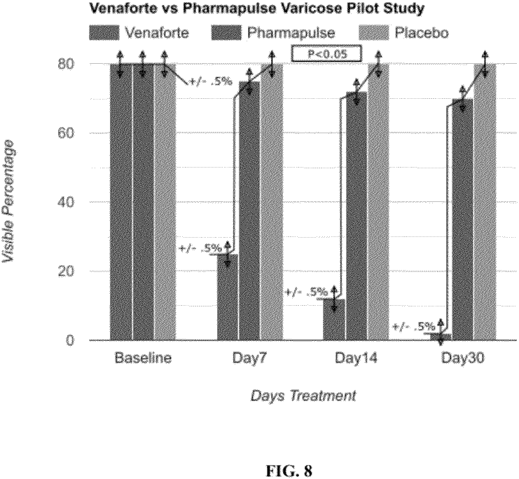

Patients were blinded to the product using a labelling scheme. For group A, as is depicted in FIG. 8, the patients using the formulation of Example 10 showed a reduction of ~80% in visibility and size of the spider veins and varicose veins (veins ranged in size from 2 mm - 4 mm). Patients in group A also noticed a reduction in discomfort from the increase in microcirculation helping reduce the excess localized fluid retention in the area of the veins. The patients in group B, as is depicted in FIG. 8, showed no change in visibility of varicose veins or spider veins. The patients in group C also showed no change in visibility of varicose veins or spider veins.

### Embodiments

Embodiment 1: A formulation for topical treatment of a vascular condition in a subject, the formulation comprising:
a sympathomimetic agent capable of effecting vasoconstriction in the skin of the subject;
a delivery system that is effective to transport an amount of the sympathomimetic agent through the epidermis that is effective to produce vasoconstriction in the skin of the subject; and
an aqueous component.

Embodiment 2: The formulation of embodiment 1 wherein the sympathomimetic agent capable of effecting vasoconstriction is selected from the group consisting of epinephrine, norepinephrine, phenylephrine, synephrine, ephedrine, or combinations thereof.

Embodiment 3: The formulation of embodiment 2 wherein the sympathomimetic agent for vasoconstriction is present in a concentration of about 0.1 wt% to 20 wt%.

Embodiment 4: The formulation of embodiment 3 wherein the delivery system comprises a compositition selected from the group consisting of polyethylene glycol (PEG), ethoxydiglycol (EDG), diethylene glycol monoethyl ether (“Transcutol”), dimethyl isosorbide (DMI), dimethyl sulfoxide (DMSO), sodium lauryl sulfate (SDS), Polyoxyethylene (20) sorbitan monooleate (Tween 80) or combinations thereof.

Embodiment 5: The formulation of embodiment 4 wherein the delivery system is present at a concentration of about 0.1 wt% to 20 wt% of the formulation.

Embodiment 6: The formulation of embodiment 4 wherein the delivery system further comprises a composition selected from the group consisting of propanediol, hyaluronic acid, sodium hyaluronate, glycerin, propylene glycol, caprylyl glycol, butylene glycol, pentylene glycol, sodium carbomer, horse chestnut extract, aescin, vanillin, xanthan gum, hydroxypropyl methylcellulose, Disodium EDTA, Tetrasodium EDTA, phenoxyethanol, ethylhexylglycerin, benzyl alcohol, diazolidinyl urea, iodopropynyl butylcarbamate DMDM hydantoin, paraben, propyl paraben, methylparaben, and polyacrylate crosspolymer or combinations thereof.

Embodiment 7: The formulation of embodiment 6 wherein the delivery system is present at a concentration of at about 1 wt% to 60 wt%.

Embodiment 8: The formulation of embodiment 4 wherein said aqueous component is present at a concentration of about 1 wt% to 60 wt%.

Embodiment 9: The formulation of embodiment 4 wherein the formulation is effective to topically treat one or more of hemorrhoids, varicose veins, spider veins, rosacea and periorbital edema.

Embodiment 10: A method of topically treating a vascular condition comprising the step of contacting skin with a formulation according to any one of embodiments 1-9.

Embodiment 11: The method of embodiment 10 wherein the epidermis is penetrated by the sympathomimetic agent to a depth of between about 1-5 millimeters.

Embodiment 12: The method of embodiment 11 wherein the formulation is applied twice daily for about 20 days for treatment of hemorrhoids.

Embodiment 13: The method of embodiment 11 wherein the formulation is applied once daily for about 30 days for treatment of spider veins.

Embodiment 14: The method of embodiment 11 wherein the formulation is applied once daily for about 35 days for treatment of varicose veins.

Embodiment 15: The method of embodiment 11 wherein the formulation is applied serially as needed for treatment of rosacea.

Embodiment 16: The method of embodiment 11 wherein the formulation is applied serially as needed for treatment of periorbital edema.

Embodiment 17: A method of preparing a formulation for topical treatment of a vascular condition comprising the steps of:
(i) dissolving a sympathomimetic agents in a niacinamide serum;
(ii) combining the product of step (i) with a penetration enhancing agent;
(iii) combining the product of step (ii) with a humectant and emollient;
(iv) combining the product of step (iii) with a preservative agent; and
(v) combining the product of step (iv) with a hydrated hyaluronic acid.

Embodiment 18: The method of embodiment 17 wherein said hyaluronic acid is partially hydrated prior to addition to the formulation.

Embodiment 19: The method of embodiment 17 further comprising the step of:

(vi) stirring the product of step (v) vigorously until a clear, slightly viscous solution is obtained.

Embodiment 20: The method of embodiment 17 wherein the amount of sympathomimetic dissolved in the formulation for treatment of periorbital edema is at a concentration of least about 0.1 wt% and at most about 5 wt%.

Embodiment 21: A formulation for topical treatment of a vascular condition in a subject, the formulation comprising: 0.1 wt% Disodium EDTA, 8 wt% Niacinamide, 10 wt% p-Synephrine HCL, 3 wt% Glycerin, 10 wt% Butylene Glycol, 10 wt% Ethoxydiglycol, 0.5 wt% Sodium Carbomer, 0.8 wt% Benzyl Alcohol, and 0.1 wt% Ethylhexylglycerin.

Embodiment 22: A formulation for topical treatment of a vascular condition in a subject, the formulation comprising: 4 wt% Epinephrine, 8 wt% Dimethyl isosorbide, 10 wt% Niacinamide, 5 wt% Glycerin, 1 wt% Vanillin, 10 wt% Propylene Glycol, 0.1 wt% Tetrasodium EDTA, 0.2 wt% diazolidinyl urea, and 0.4 wt% Xanthan gum.

Embodiment 23: A formulation for topical treatment of a vascular condition in a subject, the formulation comprising: 12 wt% D-Synephrine HCL, 15 wt% Dimethyl isosorbide, 1 wt% Vanillin, 2 wt%Glycerin, 6 wt% Pentylene glycol, 0.7 wt% Benzyl alcohol, 0.1 wt% Disodium EDTA, and 0.6 wt% Sodium hyaluronate.

Embodiment 24: A formulation for topical treatment of a vascular condition in a subject, the formulation comprising: 6 wt% L-Synephrine, 6 wt% Dimethyl isosorbide, 8 wt% Niacinamide, 1 wt% Glycerin, 8 wt% Butylene glycol, 0.05 wt% Disodium EDTA, 0.7 wt% Hyaluronic acid, 0.3 wt% Propylparaben, 0.5 wt% Methylparaben.

Embodiment 25: A formulation for topical treatment of a vascular condition in a subject, the formulation comprising: 8 wt% Synephrine, 10 wt% Dimethyl isosorbide, 8 wt% Niacinamide, 2 wt% Glycerin, 10 wt% Propanediol, 0.6 wt% Phenoxyethanol, 0.4 wt% Caprylyl glycol, 0.05 wt% Disodium EDTA, and 0.5 wt% hydroxypropyl methylcellulose.

Embodiment 26: A formulation for topical treatment of a vascular condition in a subject, the formulation comprising: 5 wt% Synephrine, 8 wt% Ethoxydiglycol, 4 wt% Niacinamide, 4 wt% Glycerin, 0.3 wt% Caprylyl glycol, 0.9 wt% Phenoxyethanol, 10 wt% Propanediol, 0.1 wt% Disodium EDTA, and 0.6 wt% polyacrylate crosspolymer.

Embodiment 27: A formulation for topical treatment of a vascular condition in a subject, the formulation comprising: 0.1 wt% Disodium EDTA, 8 wt% Niacinamide, 10 wt% p-Synephrine HCL, 3 wt% Glycerin, 10 wt% Butylene Glycol, 10 wt% Ethoxydiglycol, 0.5 wt% Sodium Carbomer, 0.8 wt% Benzyl Alcohol, and 0.2 wt% DMDM Hydantoin.

Embodiment 28: A formulation for topical treatment of a vascular condition in a subject, the formulation comprising: 4 wt% Epinephrine, 8 wt% Dimethyl isosorbide, 10 wt% Niacinamide, 5 wt% Glycerin, 1 wt% Vanillin, 10 wt% Propylene Glycol, 0.1 wt% Tetrasodium EDTA, 0.2 wt% DMDM hydantoin, and 0.4 wt% Xanthan gum.

Embodiment 29: A formulation for topical treatment of a vascular condition in a subject, the formulation comprising: 0.05 wt% Disodium EDTA, 8 wt% Niacinamide, 5 wt% Synephrine HCL, 10 wt% Dimethyl isosorbide, 10 wt% Propanediol, 3 wt% Glycerin, 0.6 wt% Phenoxyethanol, 0.1 wt% Caprylyl glycol, and 0.8 wt% Hyaluronic acid.

Embodiment 30: A formulation for topical treatment of a vascular condition in a subject, the formulation comprising: 15 wt% P-Synephrine HCL, 15 wt% Dimethyl Isosorbide, 10 wt% Propanediol, 8 wt% Niacinamide, 0.3 wt% Caprylyl Glycol, 0.7 wt% Phenoxyethanol, 0.05 wt% Disodium EDTA, and 0.5 wt% Hyaluronic acid.

### Cited Documents

Topical adrenaline in the control of intraoperative bleeding in adenoidectomy: a randomised, controlled trial- Clin Otolaryngol . 2006 Aug;31(4):303-9.

Int Forum Allergy Rhinol . 2016 Feb;6(2):135-9

Adrenaline chloride solution-epinephrine nasal solution, 1 mg/ml, for topical application-CVS pharmacy.

IM v. SC Adrenaline/EpiPen-https://www.jacionline.org/article/S0091-6749(02)70198-X/fulltexthttps://www.preparationh.com/frequently-asked-questions/ Hemorrhoids are a painful condition that affects millions of men and women. Hemorrhoids refer to a condition where the veins in the lower rectum and around the anus are swollen, dilated and inflamed (similar to varicose veins in legs).

Haq A, Michniak-Kohn B. Effects of solvents and penetration enhancers on transdermal delivery of thymoquinone: permeability and skin deposition study. Drug Deliv. 2018;25(1):1943-1949. doi:10.1080/10717544.2018.1523256

Som I, Bhatia K, Yasir M. Status of surfactants as penetration enhancers in transdermal drug delivery. J Pharm Bioallied Sci. 2012;4(1):2-9. doi:10.4103/0975-7406.92724

Osborne, D.W., Musakhanian, J. Skin Penetration and Permeation Properties of Transcutol®-Neat or Diluted Mixtures. AAPS PharmSciTech 19, 3512-3533 (2018). https://doi.org/10.1208/s12249-018-1196-8

Banov L, Knoepp LF, Erdman LH, Alia RT (1985). “Management of hemorrhoidal disease”. J S C Med Assoc. 81 (7): 398-401

Varicose veins: diagnosis and management. Clinical Guideline. National Institute for Health and Care Excellence. Jul. 24, 2014.

Sullivan DW Jr, Gad SC, Julien M. A review of the nonclinical safety of Transcutol®, a highly purified form of diethylene glycol monoethyl ether (DEGEE) used as a pharmaceutical excipient. Food Chem Toxicol. 2014 Oct;72:40-50. doi: 10.1016/j.fct.2014.06.028. Epub 2014 Jul 9. PMID: 25016034.

All documents cited in this application are hereby incorporated by reference as if recited in full herein.

Although illustrative embodiments of the present disclosure have been described herein, it should be understood that the disclosure is not limited to those described, and that various other changes or modification may be made by one of ordinary skill in the art without departing from the scope or spirit of the invention.

What is claimed is:

1. A formulation for topical treatment of a vascular condition in a subject, the formulation comprising:
a sympathomimetic agent capable of effecting vasoconstriction in the skin of the subject;
a delivery system that is effective to transport an amount of the sympathomimetic agent through the epidermis that is effective to produce vasoconstriction in the skin of the subject; and
an aqueous component.

2. The formulation of claim 1 wherein the sympathomimetic agent capable of effecting vasoconstriction is selected from the group consisting of epinephrine, norepinephrine, phenylephrine, synephrine, ephedrine, or combinations thereof.

3. The formulation of claim 2 wherein the sympathomimetic agent for vasoconstriction is present in a concentration of about 0.1 wt% to 20 wt%.

4. The formulation of claim 3 wherein the delivery system comprises a composition selected from the group consisting of polyethylene glycol (PEG), ethoxydiglycol (EDG), diethylene glycol monoethyl ether (“Transcutol”), dimethyl isosorbide (DMI), dimethyl sulfoxide (DMSO), sodium lauryl sulfate (SDS), Polyoxyethylene (20) sorbitan monooleate (Tween 80) or combinations thereof.

5. The formulation of claim 4 wherein the delivery system is present at a concentration of about 0.1 wt% to 20 wt% of the formulation.

6. The formulation of claim 4 wherein the delivery system further comprises a composition selected from the group consisting of propanediol, hyaluronic acid, sodium hyaluronate, glycerin, propylene glycol, caprylyl glycol, butylene glycol, pentylene glycol, sodium carbomer, horse chestnut extract, aescin, vanillin, xanthan gum, hydroxypropyl methylcellulose, Disodium EDTA, Tetrasodium EDTA, phenoxyethanol, ethylhexylglycerin, benzyl alcohol, diazolidinyl urea, iodopropynyl butylcarbamate DMDM hydantoin, paraben, propyl paraben, methylparaben, and polyacrylate crosspolymer or combinations thereof.

7. The formulation of claim 6 wherein the delivery system is present at a concentration of about 1 wt% to 60 wt%.

8. The formulation of claim 4 wherein said aqueous component is present at a concentration of about 1 wt% to 60 wt%.

9. The formulation of claim 4 wherein the formulation is effective to topically treat one or more of hemorrhoids, varicose veins, spider veins, rosacea and periorbital edema.

10. A method of topically treating a vascular condition comprising the step of contacting skin with a formulation according to claim 1.

11. The method of claim 10 wherein the epidermis is penetrated by the sympathomimetic agent to a depth of between about 1-5 millimeters.

12. The method of claim 11 wherein the formulation is applied twice daily for about 20 days for treatment of hemorrhoids.

13. The method of claim 11 wherein the formulation is applied once daily for about 30 days for treatment of spider veins.

14. The method of claim 11 wherein the formulation is applied once daily for about 35 days for treatment of varicose veins.

15. The method of claim 11 wherein the formulation is applied serially as needed for treatment of rosacea.

16. The method of claim 11 wherein the formulation is applied serially as needed for treatment of periorbital edema.

17. A method of preparing a formulation for topical treatment of a vascular condition comprising the steps of:
(i) dissolving a sympathomimetic agents in a niacinamide serum;
(ii) combining the product of step (i) with a penetration enhancing agent;
(iii) combining the product of step (ii) with a humectant and emollient;
(iv) combining the product of step (iii) with a preservative agent; and
(v) combining the product of step (iv) with a hydrated hyaluronic acid.

18. The method of claim 17 wherein said hyaluronic acid is partially hydrated prior to addition to the formulation.

19. The method of claim 17 further comprising the step of:
(vi) stirring the product of step (v) vigorously until a clear, slightly viscous solution is obtained.

20. The method of claim 17 wherein the amount of sympathomimetic dissolved in the formulation for treatment of periorbital edema is at a concentration of least about 0.1 wt% and at most about 5 wt%.

21. The formulation of claim 1 comprising: 0.1 wt% Disodium EDTA, 8 wt% Niacinamide, 10 wt% p-Synephrine HCL, 3 wt% Glycerin, 10 wt% Butylene Glycol, 10 wt% Ethoxydiglycol, 0.5 wt% Sodium Carbomer, 0.8 wt% Benzyl Alcohol, and 0.1 wt% Ethylhexylglycerin.

22. The formulation of claim 1 comprising: 4 wt% Epinephrine, 8 wt% Dimethyl isosorbide, 10 wt% Niacinamide, 5 wt% Glycerin, 1 wt% Vanillin, 10 wt% Propylene Glycol, 0.1 wt% Tetrasodium EDTA, 0.2 wt% diazolidinyl urea, and 0.4 wt% Xanthan gum.

23. The formulation of claim 1 comprising: 12 wt% D-Synephrine HCL, 15 wt% Dimethyl isosorbide, 1 wt% Vanillin, 2 wt%_Glycerin, 6 wt% Pentylene glycol, 0.7 wt% Benzyl alcohol, 0.1 wt% Disodium EDTA, and 0.6 wt% Sodium hyaluronate.

24. The formulation of claim 1 comprising: 6 wt% L-Synephrine, 6 wt% Dimethyl isosorbide, 8 wt% Niacinamide, 1 wt% Glycerin, 8 wt% Butylene glycol, 0.05 wt% Disodium EDTA, 0.7 wt% Hyaluronic acid, 0.3 wt% Propyl_paraben, 0.5 wt% Methylparaben.

25. The formulation of claim 1 comprising: 8 wt% Synephrine, 10 wt% Dimethyl isosorbide, 8 wt% Niacinamide, 2 wt% Glycerin, 10 wt% Propanediol, 0.6 wt% Phenoxyethanol, 0.4 wt% Caprylyl glycol, 0.05 wt% Disodium EDTA, and 0.5 wt% hydroxypropyl methylcellulose.

26. The formulation of claim 1 comprising: 5 wt% Synephrine, 8 wt% Ethoxydiglycol, 4 wt% Niacinamide, 4 wt% Glycerin, 0.3 wt% Caprylyl glycol, 0.9 wt% Phenoxyethanol, 10 wt% Propanediol, 0.1 wt% Disodium EDTA, and 0.6 wt% polyacrylate crosspolymer.

27. The formulation of claim 1 comprising: 0.1 wt% Disodium EDTA, 8 wt% Niacinamide, 10 wt% p-Synephrine HCL, 3 wt% Glycerin, 10 wt% Butylene Glycol, 10 wt% Ethoxydiglycol, 0.5 wt% Sodium Carbomer, 0.8 wt% Benzyl Alcohol, and 0.2 wt% DMDM Hydantoin.

28. The formulation of claim 1 comprising: 4 wt% Epinephrine, 8 wt% Dimethyl isosorbide, 10 wt% Niacinamide, 5 wt% Glycerin, 1 wt% Vanillin, 10 wt% Propylene Glycol, 0.1 wt% Tetrasodium EDTA, 0.2 wt% DMDM hydantoin, and 0.4 wt% Xanthan gum.

29. The formulation of claim 1 comprising: 0.05 wt% Disodium EDTA, 8 wt% Niacinamide, 5 wt% Synephrine HCL, 10 wt% Dimethyl isosorbide, 10 wt% Propanediol, 3 wt% Glycerin, 0.6 wt% Phenoxyethanol, 0.1 wt% Caprylyl glycol, and 0.8 wt% Hyaluronic acid.

30. The formulation of claim 1 comprising: 15 wt% P-Synephrine HCL, 15 wt% Dimethyl Isosorbide, 10 wt% Propanediol, 8 wt% Niacinamide, 0.3 wt% Caprylyl Glycol, 0.7 wt% Phenoxyethanol, 0.05 wt% Disodium EDTA, and 0.5 wt% Hyaluronic acid.
